(19) United States
(12) Patent Application Publication
(10) Pub. No.: US 2009/0312653 A1
(43) Pub. Date: Dec. 17, 2009

(54) Method and apparatus for determining cardiac medical parameters from supra-systolic signals obtained from an oscillometric blood pressure system

(76) Inventors: Nigel E. Sharrock, New York, NY (US); Andrew Lowe, Auckland (NZ)

Correspondence Address: MILDE & HOFFBERG, LLP, 10 BANK STREET, SUITE 460, WHITE PLAINS, NY, 10606, US

(21) Appl. No.: 12/455,516

(22) Filed: Jun. 3, 2009

Related U.S. Application Data
(60) Provisional application No. 61/132,120, filed on Jun. 16, 2008.

(51) Int. Cl. A61B 5/02

(52) U.S. Cl. 600/495

(57) ABSTRACT

A method and apparatus are disclosed for determining certain cardiac medical parameters that are useful is diagnosing and treating cardiovascular disease. The apparatus is designed to carry out the method, which includes the steps of:
(a) inflating a blood pressure cuff on the brachial artery to a supra-systolic pressure;
(b) sensing a sequence of pressure pulse waveforms associated with the brachial artery that result from a plurality of cardiac ejection cycles;
(c) averaging the waveforms to produce an average, representative waveform having an initial, incident wave portion and a reflected wave portion;
(d) analyzing the representative waveform to determine a value of one or more cardiac medical parameters including the reflection wave ratio (RWR), the reflected wave transit time (RWTT), the maximum amplitude of the initial pressure wave (PS1), the maximum rise (slope) of the initial pressure wave (dp/dt), and/or the systolic ejection period (SEP); and
(e) displaying the value of the medical parameter(s).

### CROSS-REFERENCE TO RELATED APPLICATIONS

This present application claims benefit of priority from U.S. patent application Ser. No. 11/358,283, filed Feb. 21, 2006 (now U.S. Patent Publication No. 2006/0224070-A1, published Oct. 5, 2006); U.S. patent application Ser. No. 12/157,854, filed Jun. 13, 2008 (now U.S. Patent Publication No. 2009/0012411-A1, published Jan. 8, 2009) and U.S. Provisional Application Ser. No. 61/132,120, filed Jun. 16, 2008. The invention disclosed and claimed herein is related in subject matter to that disclosed in U.S. Pat. No. 5,913,826, issued Jun. 22, 1999; U.S. Pat. No. 6,994,675, issued Feb. 7, 2006; and the aforementioned U.S. Patent Publication No. 2006/0224070-A1 and U.S. Patent Publication No. 2009/0012411-A1, all of which are incorporated herein by reference.

### BACKGROUND OF THE INVENTION

Blood pressure is the net result of stroke volume and vascular resistance or impedance. Blood pressure can increase with an increase in stroke volume as occurs with exercise or with adrenaline. Blood pressure can also increase with an increase in arterial tone, which is the usual cause of essential hypertension. Blood pressure increases with vasoconstrictors such as phenylephrine or angiotensin which raise blood pressure solely by increasing vascular stiffness.

It would be very useful to be able to quantify the relative contribution of stroke volume and arterial stiffness to blood pressure. For example, if the oscillometrically measured blood pressure is 150/80, are these high numbers due to increases in stroke volume or from arterial stiffness? The decision to treat or not to treat, and/or the determination of what agent to use, could vary, depending upon the result.

Similarly, the response to the treatment to be followed can vary with the result. For example, if a vasodilator such as an angiotensin receptor blocker (ARB) is used, the change in vascular stiffness may be more important to follow, rather than blood pressure alone, as arterial stiffness is the primary pathology.

In the acute care setting, a non-invasive measure would help in decision-making to diagnose and manage heart failure or sepsis with vasoactive drugs and fluid.

There is also a large group of people with normal blood pressure but increased vascular stiffness. A non-invasive way of assessing the degree of vasoconstriction and cardiac performance would be helpful in diagnosing and treating such patients. Other patients have unrecognized vascular stiffness yet their blood pressure does not reach the 140/90 threshold of treatment. How to treat (or not to treat) these patients is unclear. The ability to further characterize those patients who may have so-called “pre-hypertension” into those with and without vascular stiffness could provide a way forward in therapy and prevention of premature vascular death.

### SUMMARY OF THE INVENTION

The principal object of the present invention is to provide a method and apparatus for identifying the various components of the arterial pulse, and from these components to determine a cardiovascular profile, from a common non-invasive clinical test—a simple oscillometric blood pressure measurement.

Most arterial wave analysis is presently based upon intra-arterial pulse waves or waveforms obtained from Doppler or tonometry techniques. The present invention makes possible the assessment of such waveforms obtained using a normal blood pressure cuff.

This object, as well as other objects which will become apparent from the discussion that follows, are achieved, according to the present invention, by providing a method of, and apparatus for, determining a cardiovascular status of a mammal having a cardiovascular system that includes a brachial artery, by:
(a) inflating a blood pressure cuff on the brachial artery to a supra-systolic pressure;
(b) sensing a sequence of pressure pulse waveforms associated with the brachial artery that result from a plurality of cardiac ejection cycles;
(c) averaging the waveforms to produce a mean, representative waveform having an initial, incident wave portion and a reflected wave portion;
(d) analyzing the representative waveform to determine a value of one or more cardiac medical parameters including the reflection wave ratio (RWR), the reflected wave transit time (RWTT), the maximum amplitude of the initial pressure wave (PS1), the maximum rise (slope) of the initial pressure wave (dp/dt), and/or the systolic ejection period (SEP); and
(e) displaying the value of the medical parameter(s).

Apparatus is also provided for carrying out the method.

The various cardiac medical parameters are defined, and their values are determined, from the representative waveform, as follows:

Reflection Wave Ratio (RWR)

The “reflection wave ratio” provides a measure of large arterial tone or arterial stiffness. The RWR is determined as the ratio of peak amplitude of the reflected wave to that of the initial, incident wave.

Reflected Wave Transit Time (RWTT)

Measurement of the time delay between the initial, incident wave and the subsequent reflected wave provides a surrogate measure of pulse wave velocity. Changes in this value in a patient over time, or with treatment, reflect a change in pulse wave velocity and a change in large arterial stiffness or compliance. The RWTT can be assessed by measuring the start-to-start or peak-to-peak time period of the successive incident and reflection waves. The peak-to-peak time period is also called “time of reflection” (TR).

Peak Pressure (PS1) of Incident Wave

The maximum amplitude of the initial, incident wave (PS1) serves as a surrogate for cardiac stroke volume. Increases or decreases in this peak amplitude of the incident wave in a patient over time, or with therapy, represent changes in stroke volume. The measured area under the incident wave curve may also be used to measure these changes.

Maximum Rate of Increase (Slope) of PS1 (dp/dt)

The rate of change in pressure of the incident wave over time (Dp/dt), or the maximum slope of the rise in this wave, represents a change in cardiac performance or “contractility”.

Systolic Ejection Period (SEP)

The total systolic ejection period is measured from the beginning of the incident wave to the end of the reflected wave. This parameter is useful for assessing vascular compliance.

For a full understanding of the present invention, reference should now be made to the following detailed description of the preferred embodiments of the invention as illustrated in the accompanying drawings.

### BRIEF DESCRIPTION OF THE DRAWINGS

FIG. 1 is a block diagram showing the preferred embodiment of apparatus according to the invention for obtaining supra-systolic signals from a blood pressure cuff and determining from these signals certain cardiac medical parameters which are useful in diagnosing and treating cardiac disease.

FIG. 2 is a more detailed block diagram of the apparatus of FIG. 1.

FIG. 3 is a time diagram showing a single representative pulse waveform produced by averaging supra-systolic signals sensed during a plurality of cardiac ejection cycles and indicating certain cardiac medical parameters which are determined according to the invention.

FIGS. 4a and 4b are time diagrams showing pulse waveforms of a patient on 50 mg/day of Losartan (FIG. 4a) and after stopping Losartan (FIG. 4b).

FIGS. 5a and 5b are time diagrams showing pulse waveforms of a patient both before (FIG. 5a) and after (FIG. 5b) the patient's hand is inserted in ice water.

FIGS. 6a and 6b are time diagrams showing pulse waveforms of a patient both before (FIG. 6a) and after (FIG. 6b) the administration of Ephedrine, a cardiac stimulant.

FIGS. 7a and 7b are time diagrams showing pulse waveforms of a patient both before (FIG. 7a) and after (FIG. 7b) exercising on a stationary bicycle.

FIGS. 8a and 8b are time diagrams showing pulse waveforms of a patient both before (FIG. 8a) and after (FIG. 8b) the administration of Propofol, a vasodilator.

FIGS. 9a and 9b are time diagrams showing pulse waveforms of a patient both before (FIG. 9a) and after (FIG. 9b) the administration of Phenylephrine, a vasoconstrictor and cardiac depressant.

FIGS. 10a and 10b are time diagrams showing pulse waveforms of a patient both before (FIG. 10a) and after (FIG. 10b) the administration of low dose Epinephrine.

FIG. 11 is a flow chart showing the algorithm employed by the apparatus of FIGS. 1 and 2 for processing supra-systolic signals.

FIG. 12 is a flow chart showing a detailed algorithm for a section of the flow chart of FIG. 11, for pre-filtering the signal.

FIG. 13 is a flow chart showing a detailed algorithm for a section of the flow chart of FIG. 11, for normalizing and finding the commencement of heartbeats.

FIG. 14 is a flow chart showing a detailed algorithm for a section of the flow chart of FIG. 11, for roughly aligning the heartbeats.

FIG. 15 is a flow chart showing a detailed algorithm for a section of the flow chart of FIG. 11, for finely aligning the heartbeats.

FIG. 16 is a flow chart showing a detailed algorithm for a section of the flow chart of FIG. 11, for calculating the average heartbeat.

FIG. 17 is a flow chart showing the algorithm, which is a continuation of the algorithm in the flow chart of FIG. 11, for finding points on an average heartbeat.

FIG. 18 is a flow chart showing a detailed algorithm for a section of the flow chart of FIG. 17, for finding the foot of a heartbeat.

FIG. 19 is a flow chart showing a detailed algorithm for a section of the flow chart of FIG. 17, for finding the peak of the SS1 wave.

FIG. 20 is a flow chart showing a detailed algorithm for a section of the flow chart of FIG. 17, for finding the peak of the SS2 wave.

FIG. 21 is a flow chart showing a detailed algorithm for a section of the flow chart of FIG. 17, for finding the trough between the SS1 and the SS2 wave.

FIG. 22 is a flow chart showing a detailed algorithm for a section of the flow chart of FIG. 17, for finding the trough after the SS2 wave.

FIG. 23 is a flow chart showing a detailed algorithm for a section of the flow chart of FIG. 11, for finding the peak of the SS3 wave.

FIG. 24 is a time diagram showing a plurality of cardiac pulse waveforms illustrating the progressive filtering method used to obtain the featured points.

FIG. 25 is a time diagram showing a single representative pulse waveform with noise.

### DETAILED DESCRIPTION OF THE PREFERRED EMBODIMENTS

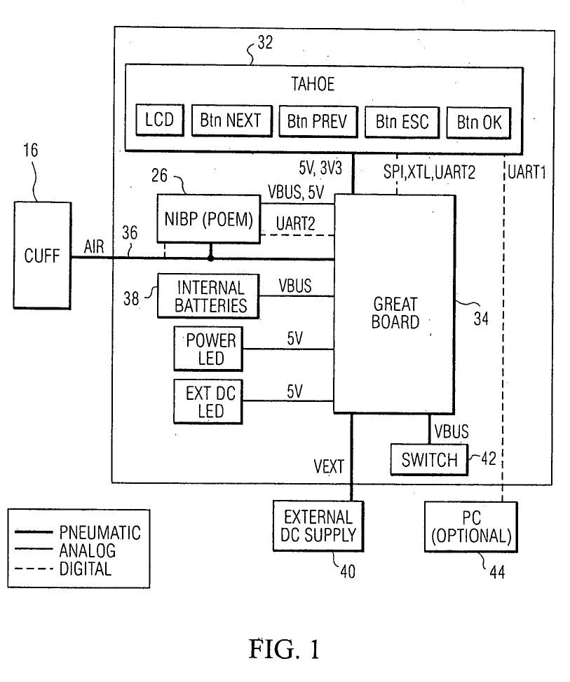

The preferred embodiments of the present invention will now be described with reference to FIGS. 1-25 of the drawings. Identical elements in the various figures are designated with the same reference numerals.

This invention concerns the measurement, processing and display of certain cardiac medical parameters obtained, using a blood pressure cuff on the brachial artery, by sensing pressure pulse waveforms with a wideband pressure transducer during a plurality (e.g. from 5 to 15) cardiac ejection cycles and taking the average.

Oscillometric Blood Pressure System

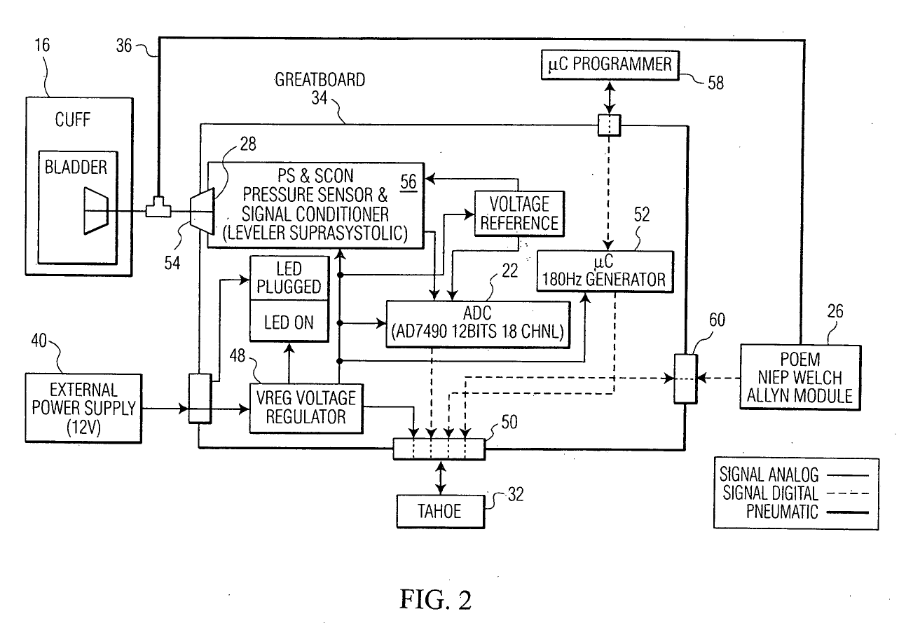

FIGS. 1 and 2 are block diagrams of a preferred embodiment of the oscillometric apparatus according to the invention. The apparatus is controlled by an embedded central processing unit (“CPU”) designated as Tahoe 32. Tahoe 32 interfaces with a “great board” 34, which in turn is connected to the other components of the apparatus. The great board 34 contains custom signal processing electronics (as further explained below), and is connected to cuff 16 by pneumatic connector 36. Pneumatic connector 36 also connects NIBP measurement module 26 which controls the pneumatic pressure in cuff 16 and achieves and maintains the proper supra-systolic pressure in cuff 16. NIBP measurement module 26 can be a commercially available unit, such as supplied by Welch Allyn under the name POEM. NIBP measurement module 26 is electronically connected to great board 34, which inputs the pre-determined supra-systolic pressure information to the module 26. As shown in FIG. 1, the apparatus contains internal batteries 38 and an external DC power supply 40, and is operated by switch 42. The apparatus can optionally be connected to a PC 44, interfaced through the Tahoe 32.

FIG. 2 illustrates further detail of the components of the great board 34. Generally, the great board 34 contains components relating to power regulation and supply 48, an interface 50 to the Tahoe board 32, an interface 60 to NIBP measurement module 26, and a 100 Hz generator 52 for pacing A/D converter 22. Also, great board 34 comprises pneumatic interface 54 for pneumatic connection through pneumatic connecter 36 to cuff 16. Pneumatic interface 54 is connected to pressure sensor 28 within great board 34, which measures the cuff pulse waves and provides a transduced analog signal to signal conditioner (“SCON”) 56. The output analog signal of SCON 56 is input into A/D converter 22 where it is converted into a digital signal and passed to the Tahoe 32. A/D converter 22 can be a 12 bit 16 channel A/D converter, such as AD7490.

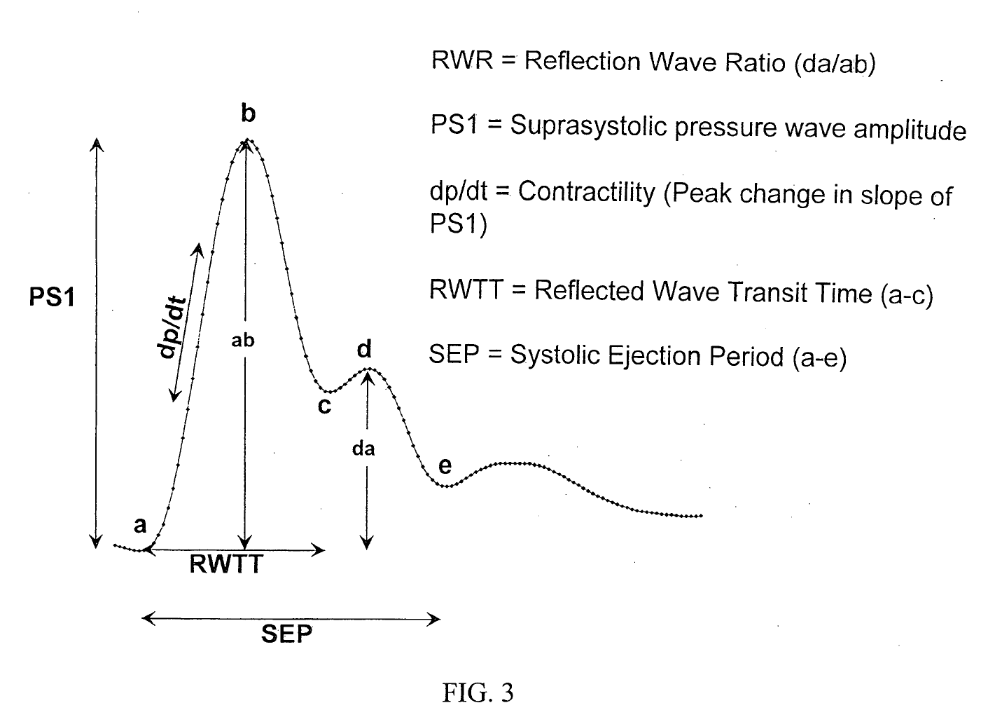

The Tahoe 32 comprises a dedicated CPU which averages the multiple digitized pulse waveform signals received from the A/D converter to produce, store and display a single, representative cadiac pulse waveform of the type shown in FIG. 3. The method of calculating this average waveform or “beat” will be described hereinbelow.

Standard Cardiac Pulse Waveform

FIG. 3 illustrates a representative waveform which is obtained by averaging a sequence of pressure pulse waveforms sensed during a succession of cardiac ejection cycles (from five to fifteen, and preferably ten or twelve) and taking the average. Various significant points on the waveform have been designated with lower case letters. Specifically, the letter a designates the initial trough at the commencement of the ejection cycle; the letter b designates the peak amplitude of the initial or incident wave; letter c designates the subsequent trough; letter d designates the peak of the second or reflected wave; and the letter e designates the trough following the reflected wave.

Determining Medical Parameters

Various cardiac medical parameters which are determined by the method and apparatus of the present invention are set forth and illustrated in FIG. 3. These are:

Reflection Wave Ratio (RWR)=(d−a)/(b−a);

Reflected Wave Transit Time (RWTT)=Tc−Ta;

Peak Pressure of Incident Pressure Wave (PS1)=b−a;

Peak slope of Incident Pressure Wave (dp/dt)=Max dp/dt;

and

Systolic Ejection Period (SEP)=Te−Ta;

where a, b, etc. are the pressures in mm Hg, and Ta, Tc and Te are times at points a, c and e, respectively.

The remaining figures illustrate how the cardiac medical parameters may be used, with and without drug therapy, to assess the cardiac performance of a patient. As will be explained below, the parameters provide useful information especially when they are determined multiple times to generate historical data.

Supra-systolic recordings can be used to characterize the physiology underlying blood pressure, which is the next result of stroke volume and large arterial impedance or vascular resistance. An increased blood pressure can be secondary to increased stroke volume or increased vascular resistance or a combination. Treatment decisions are better defined by knowing what causes the increased blood pressure. For example, if the cause is increased arterial resistance, a vasodilator is indicated. If the cause is increased stroke volume, either no treatment is indicated or a beta blocker can be used.

If someone's blood pressure is marginally elevated, due to increased arterial resistance, it may be appropriate to treat the blood pressure with a vasodilator thus providing a more rational basis upon when to treat patients with prehypertension.

Many patients with blood pressure of 130/80 kg may have increased vascular stiffness (MW RWR) and benefit from vasodilator therapy decreasing their chance of stroke, myocardial infarction and renal failure.

Similarly, low blood pressure (hypotension) can be due to a reduction in stroke volume or vasodilatation. The treatment and prognosis are therefore different. Low stroke volume and low cardiac output are poorly tolerated and cardiac stimulation is preferred. Conversely, a low blood pressure from vasodilatation is well tolerated requiring less urgent (and different) treatment.

Thus, the present invention, by measuring RWR and PS1, can be used to analyze blood pressure and improve clinical decision making in both ambulatory and critical care environments.

PS1 represents the intensity or energy of the incident waveform upon cardiac ejection. It is dependent upon both blood pressure and stroke volume. An increase in stroke volume (due, e.g., to fluid, ephedrine or epinephrine) or of blood pressure (ephedrine) increases PS1. Conversely, a reduction in stroke volume (phenylephrine plus epidural anesthesia) results in a decrease in PS1.

Thus PS1 is a measure of pulse wave amplitude and can be used to assess stroke volume if blood pressure and reflection wave ratio (RWR), a measure of vascular tone, are known.

For example, with propofol, if RWR decreases but PS1 is maintained, stroke volume should be increased. With epidural anesthesia with phenylephrine, where RWR increases and PS1 decreases, stroke volume should be decreased. PS1 increases and RWR decreases with exercise and epinephrine, therefore, stroke volume must be enhanced.

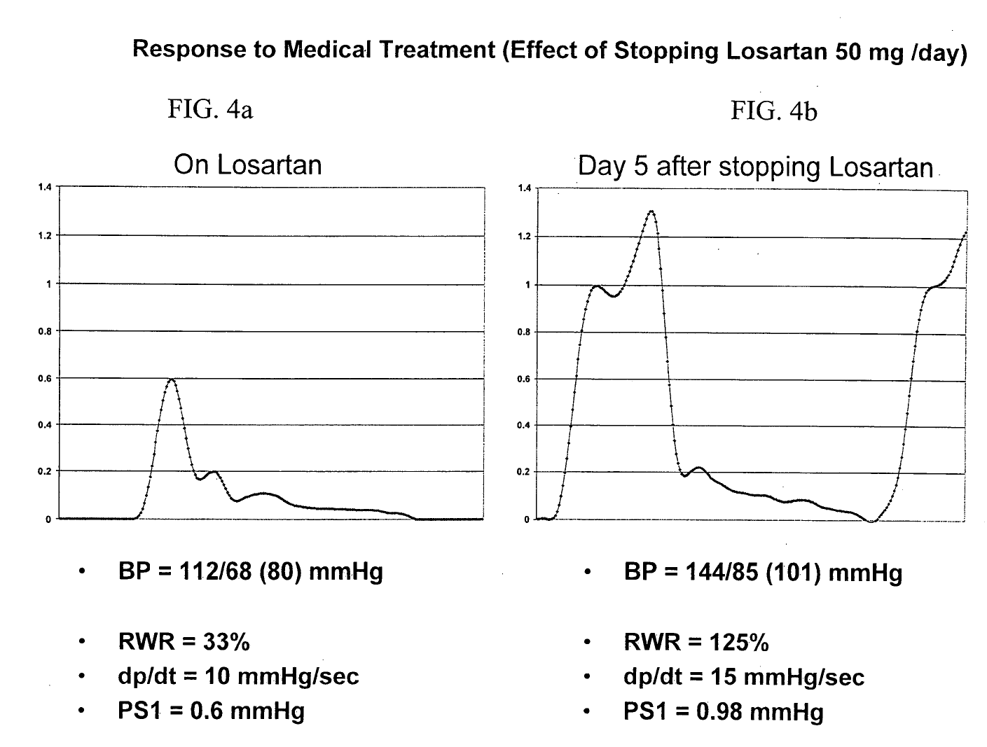

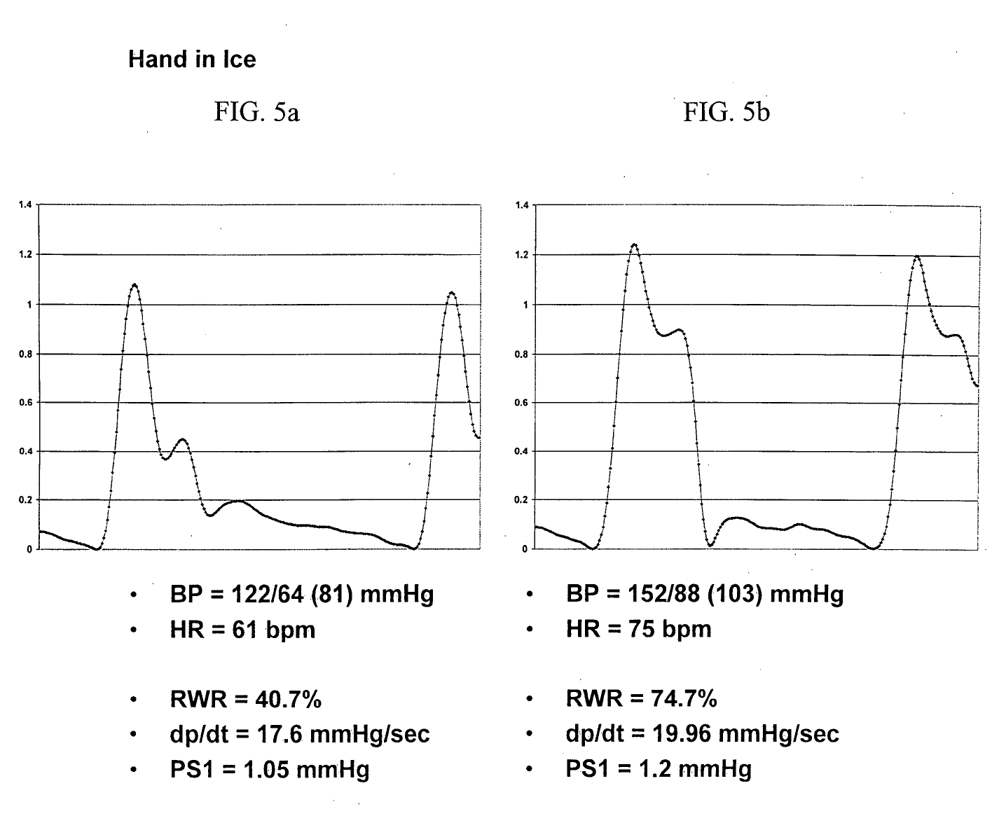

1. An example of an increase in blood pressure from vasoconstriction (increase in reflection wave ratio (RWR)) is shown with discontinuation of a vasopressor Losartan (FIG. 4) and hand in ice (FIG. 5).

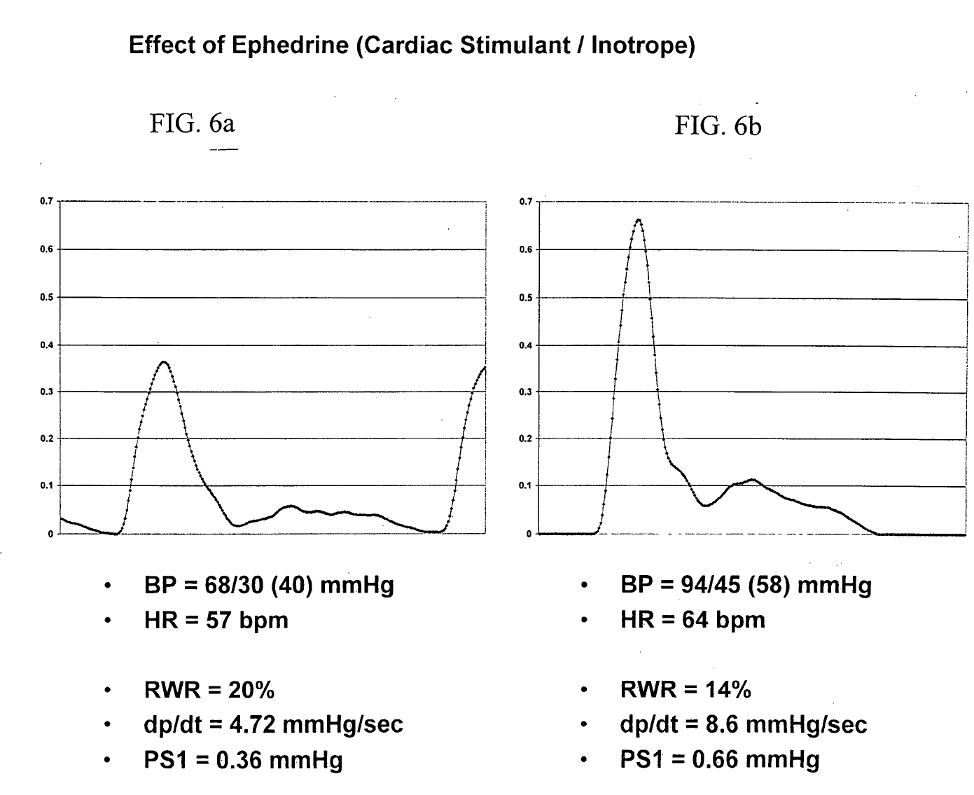

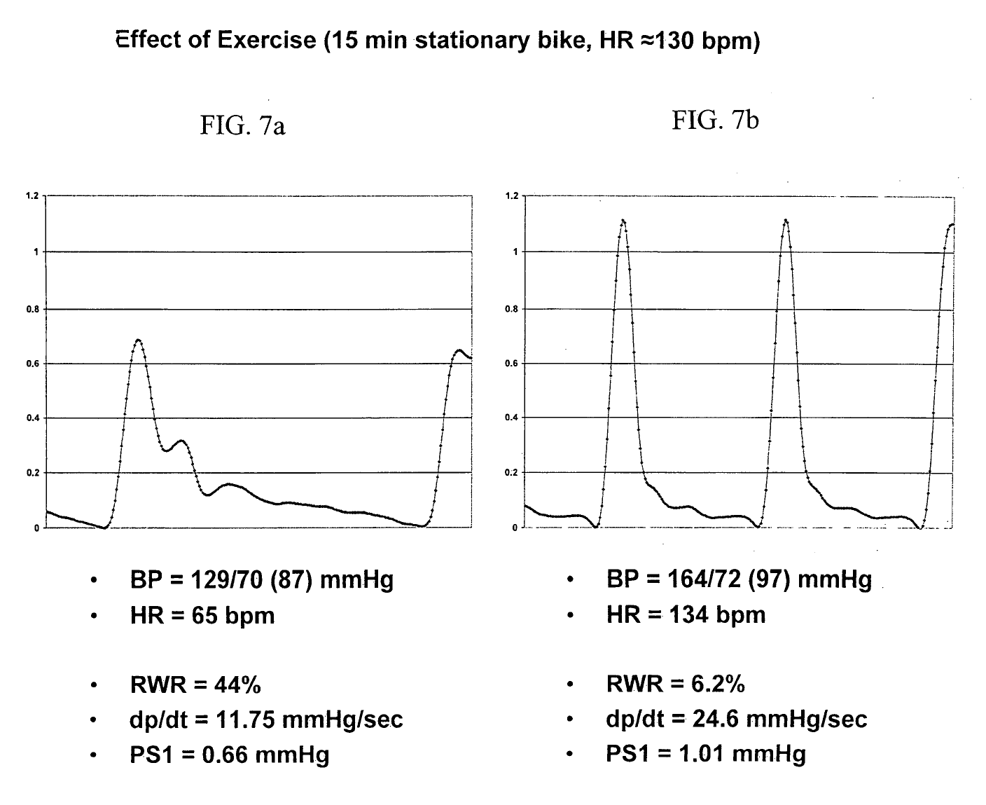

2. An example of an increase in blood pressure secondary to an increase in stroke volume, with an increase in PS1 but no increase in RWR, is shown in FIG. 6 (ephedrine) and FIG. 7 (exercise).

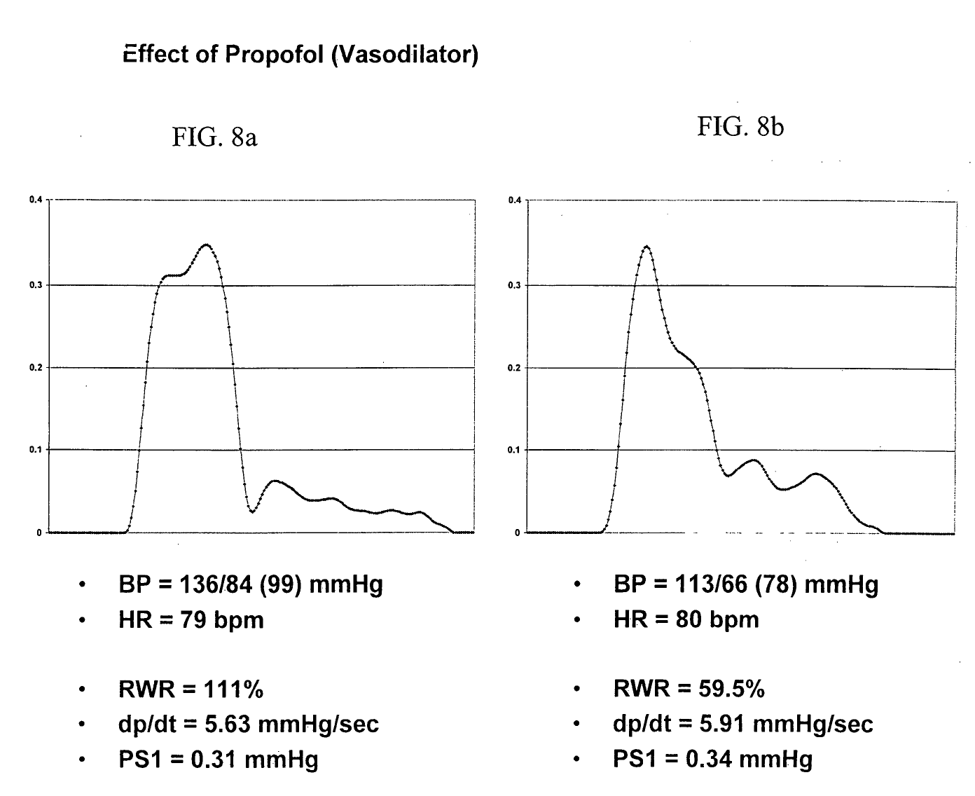

3. An example of a reduction in blood pressure due to vasodilatation is shown in FIG. 8 with vasodilatation due to propofol (and little change in PS1).

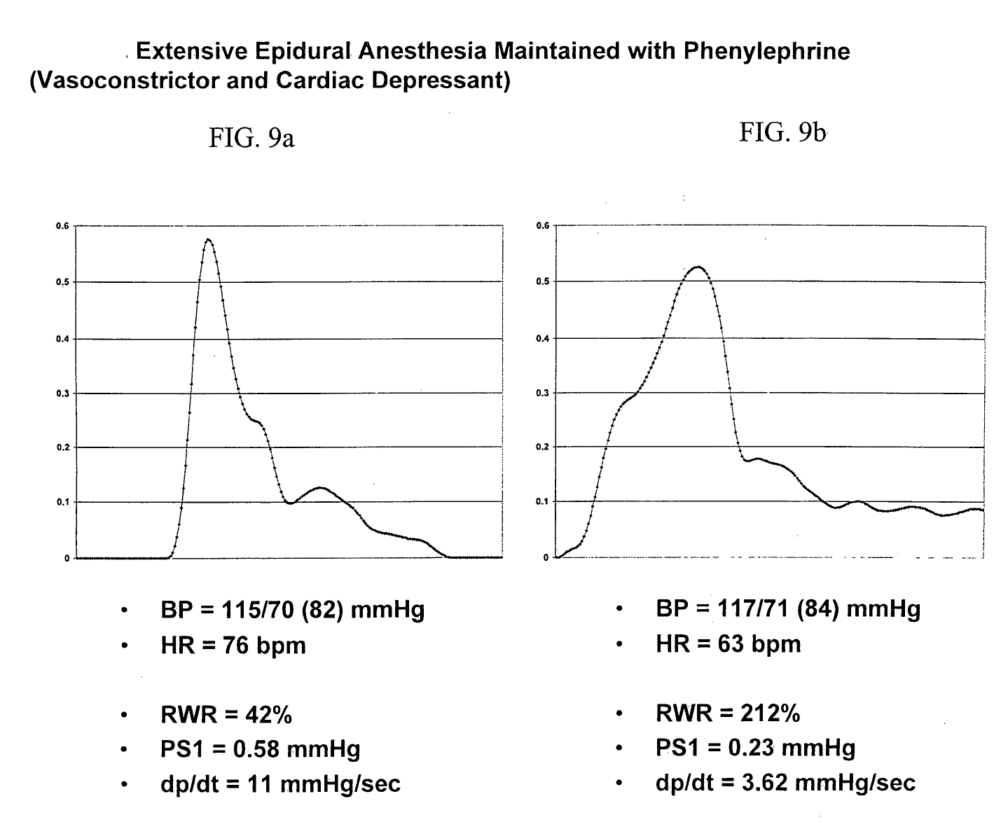

4. An example of a reduction on blood pressure due to cardiac depression is shown in FIG. 9 wherein an extensive epidural is maintained with phenylephrine. This anesthetic results in a reduction of stroke volume, resulting in a reduction in PS1 and an increase in RWR.

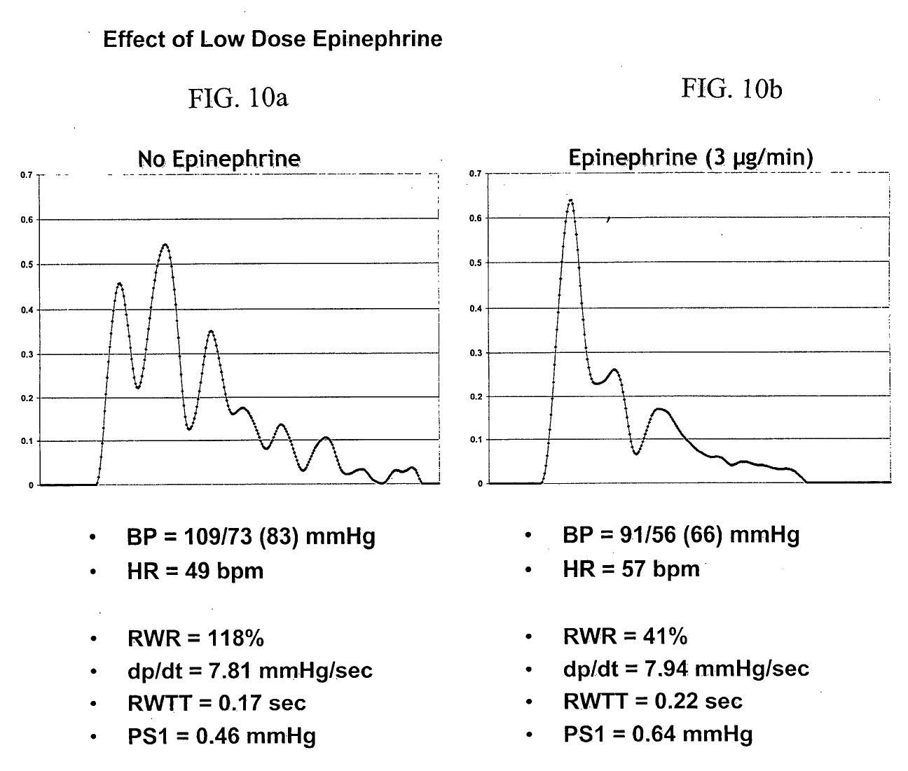

5. The effect of low dose epinephrine (FIG. 10) is associated with an increase in stroke volume and a slight reduction on blood pressure due to arterial dilatation. PS1 increases and RWR is reduced as systolic blood pressure goes from 109 to 91 mmHg. These changes are not apparent from a measurement of blood pressure alone.

Calculating an Average Pulse Waveform

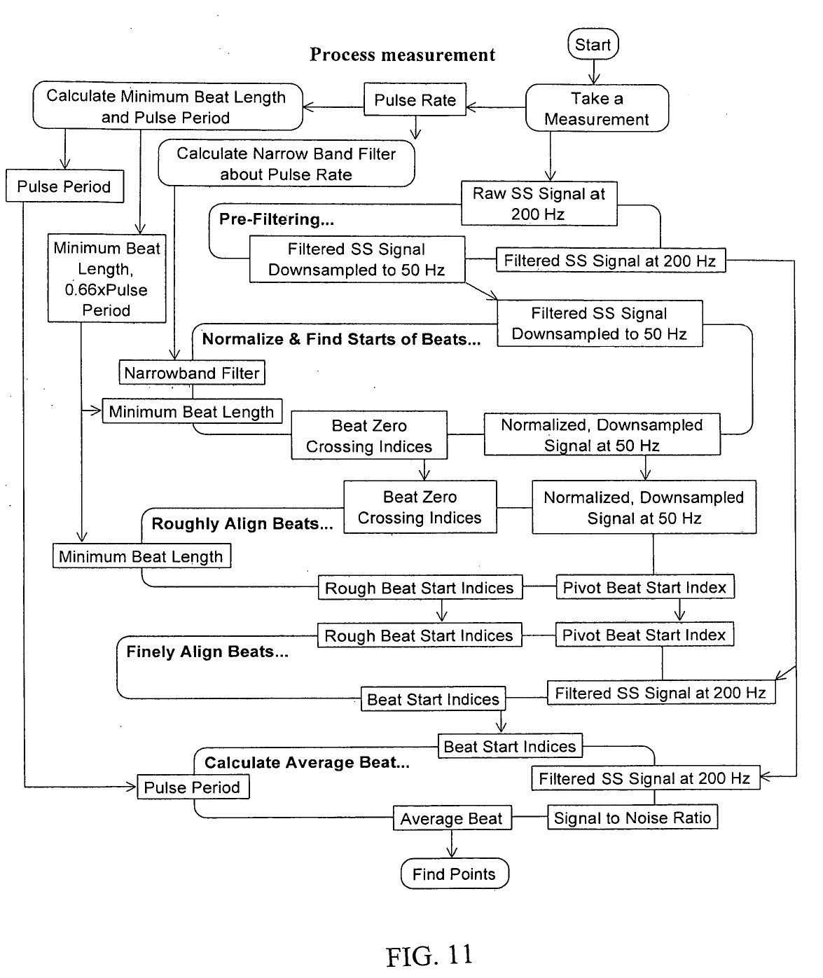

The average cardiac pulse waveform or “beat” is calculated from measurement of supra-systolic (SS) signals for approximately 10 seconds. The raw SS signal is sampled at 200 Hz. This sampled signal is then processed in the manner shown in the flowcharts of FIGS. 11-16.

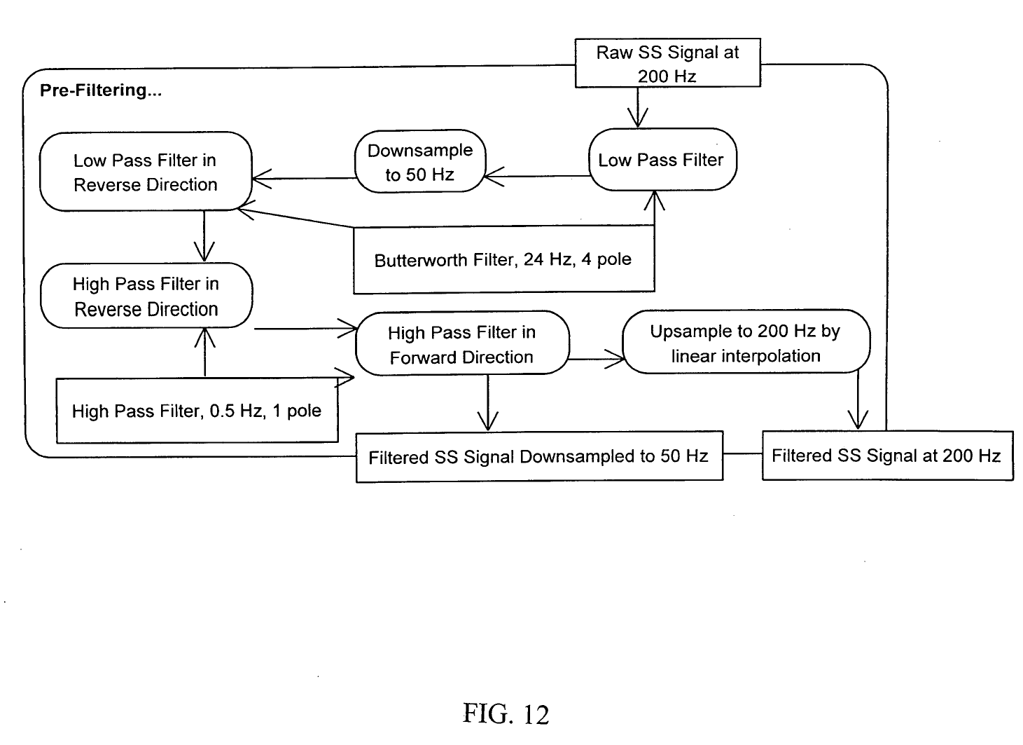

FIG. 11 is a flow chart showing the overall algorithm for calculating the average pulse waveform. Details of various portions of this algorithm are illustrated in FIGS. 12-16, respectively.

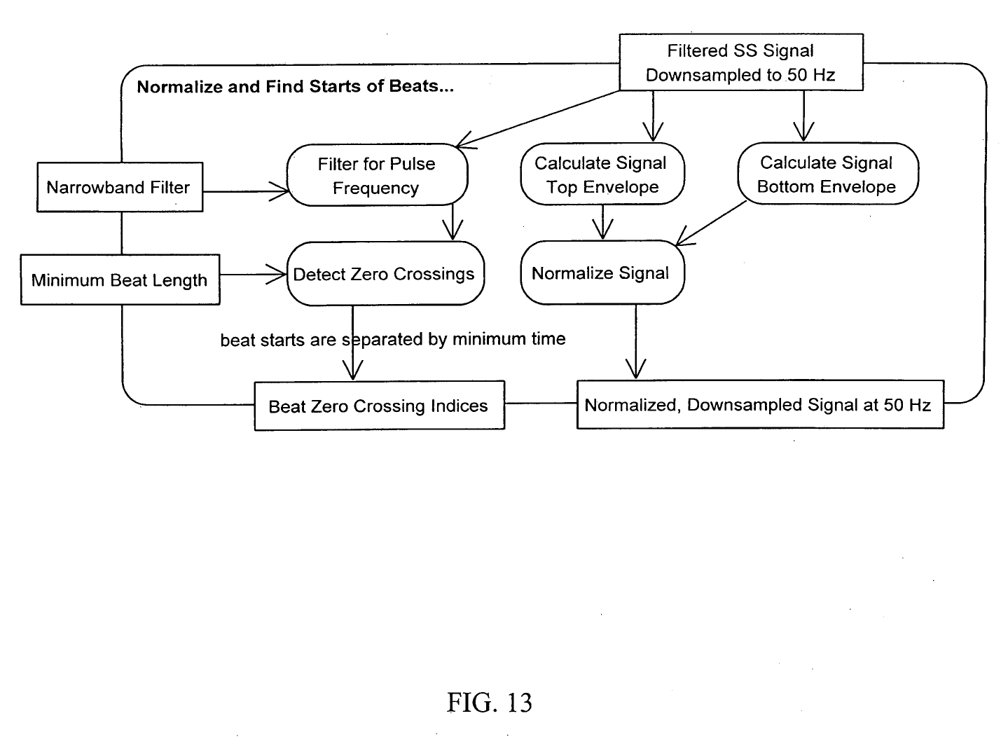

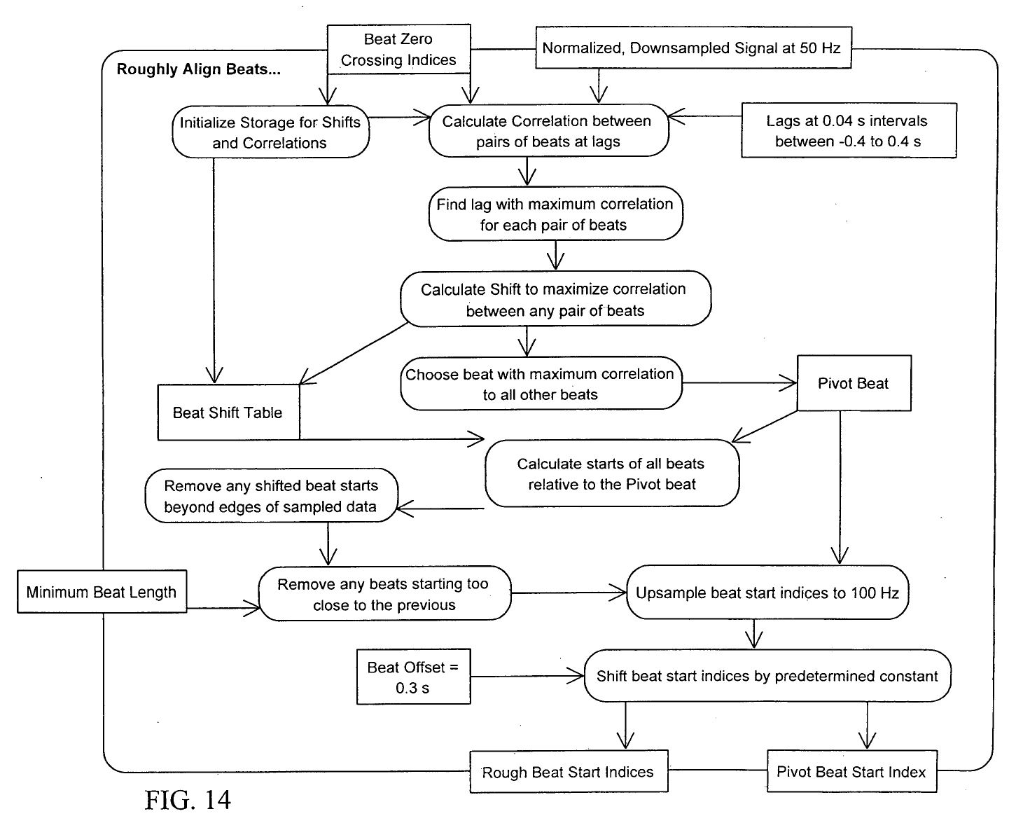

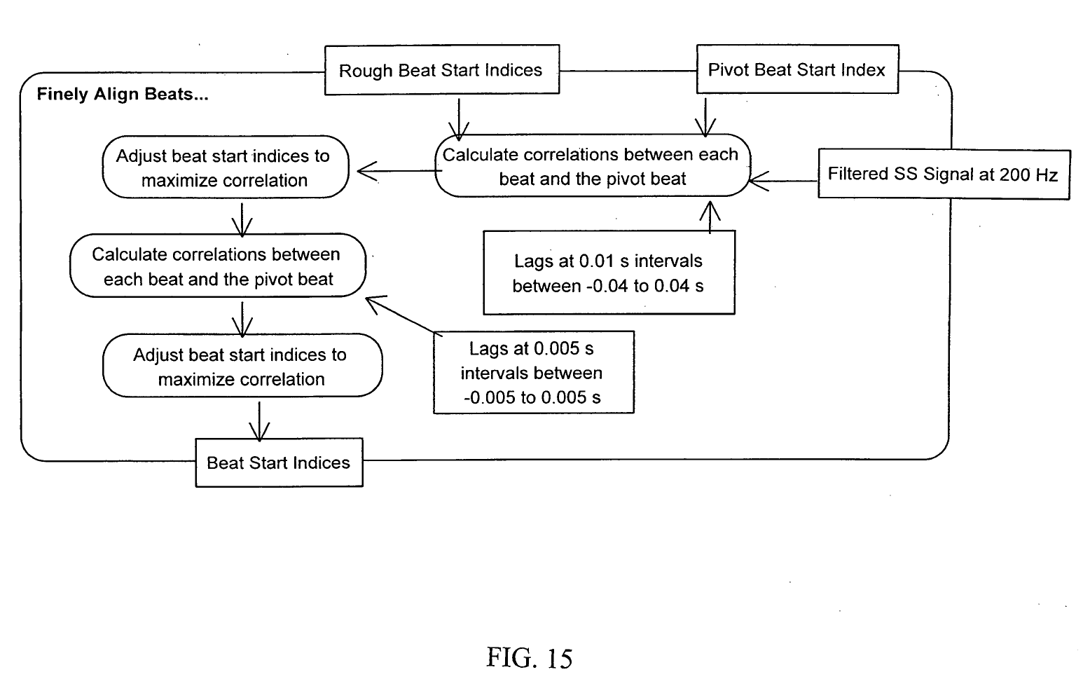

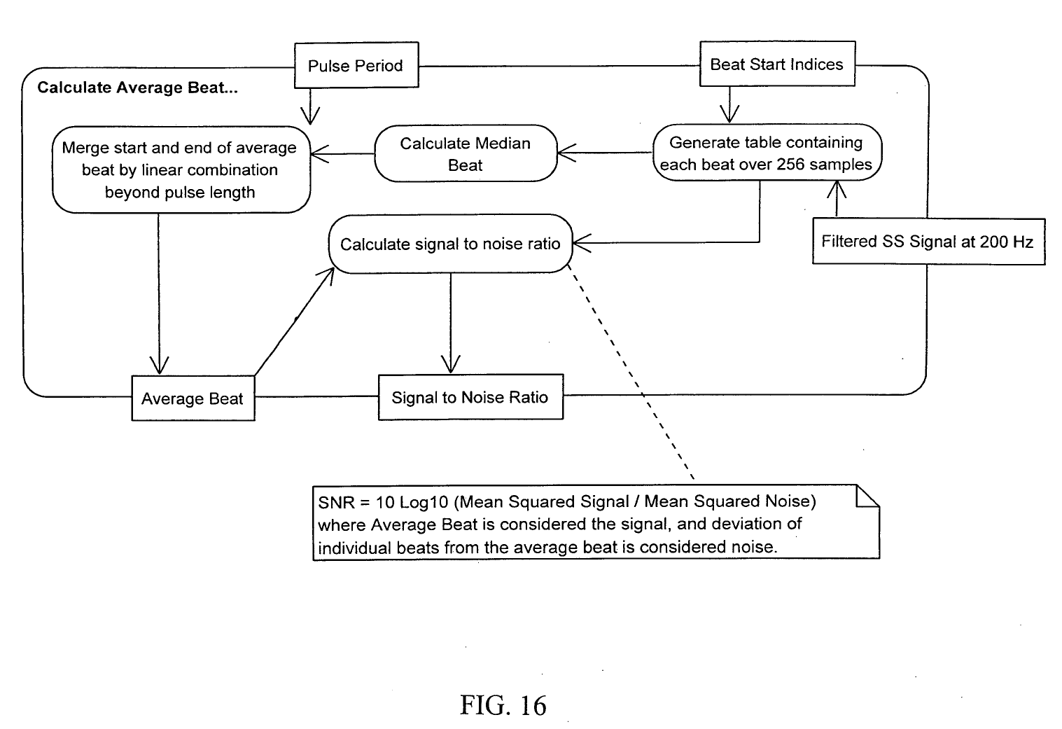

As illustrated in these figures, the process measurement proceeds as follows:
1. The signal is filtered and down-sampled to speed up processing (FIG. 12).
2. The down-sampled signal is normalized, narrow-band filtered and then individual heart beats are found from the zero crossings (FIG. 13).
3. The down-sampled beats are roughly aligned by finding the lags at which the cross correlation between beats is maximized (FIG. 14).
4. The alignment is further refined using the filtered, but not down-sampled, beats obtained in step 1 (FIG. 15).
5. The average of the aligned beats is then calculated using the median at each time point. The signal-to-noise ratio (SNR) is calculated based on the average beat (FIG. 16).

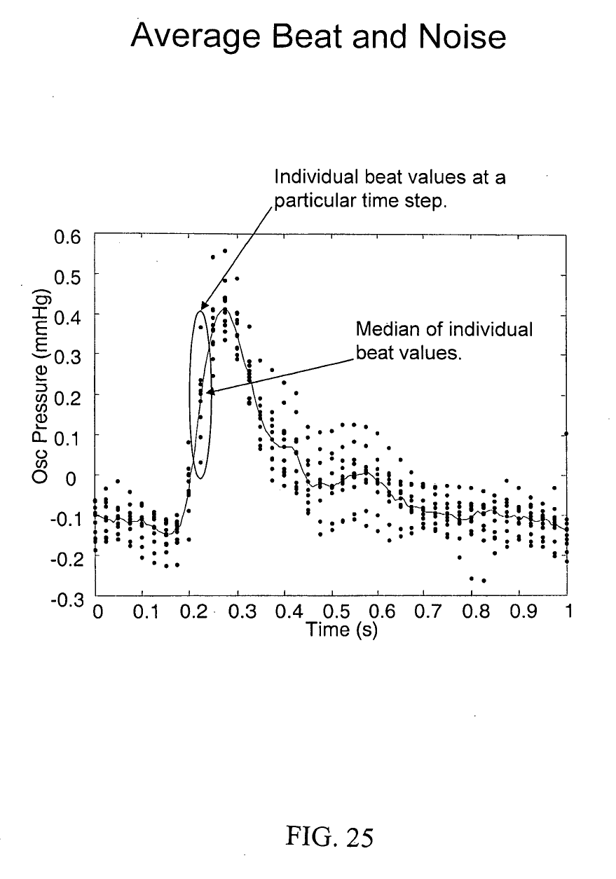

FIG. 25 illustrates the noise associated with the calculated average beat. The SNR is calculated using the following formula:

$$S N R = 10 {Log}_{10} \frac{{\sum }_{n = 1}^{N} {q}_{n}^{2}}{\frac{1}{B} {\sum }_{b = 1}^{B} {\sum }_{n = 1}^{N} {( {p}_{b , n} - {q}_{n} )}^{2}}$$

where B is the number of beats, N is the number of samples in each beat, p_{b,n} is the nth sample on beat b, and qₙ is the nth sample on the average beat.

The SNR is used to gauge the quality of the processed signal. If the patient moves, or experiences arrhythmias, the SNR will decrease. If all the beats are very similar to the average beat, the SNR is high. Generally, a good signal has an SNR greater than 12 dB, whereas a poor signal has an SNR less than 3 dB.

The SNR of the signal is preferably displayed to the user as a cue to the reliability of the measurement.

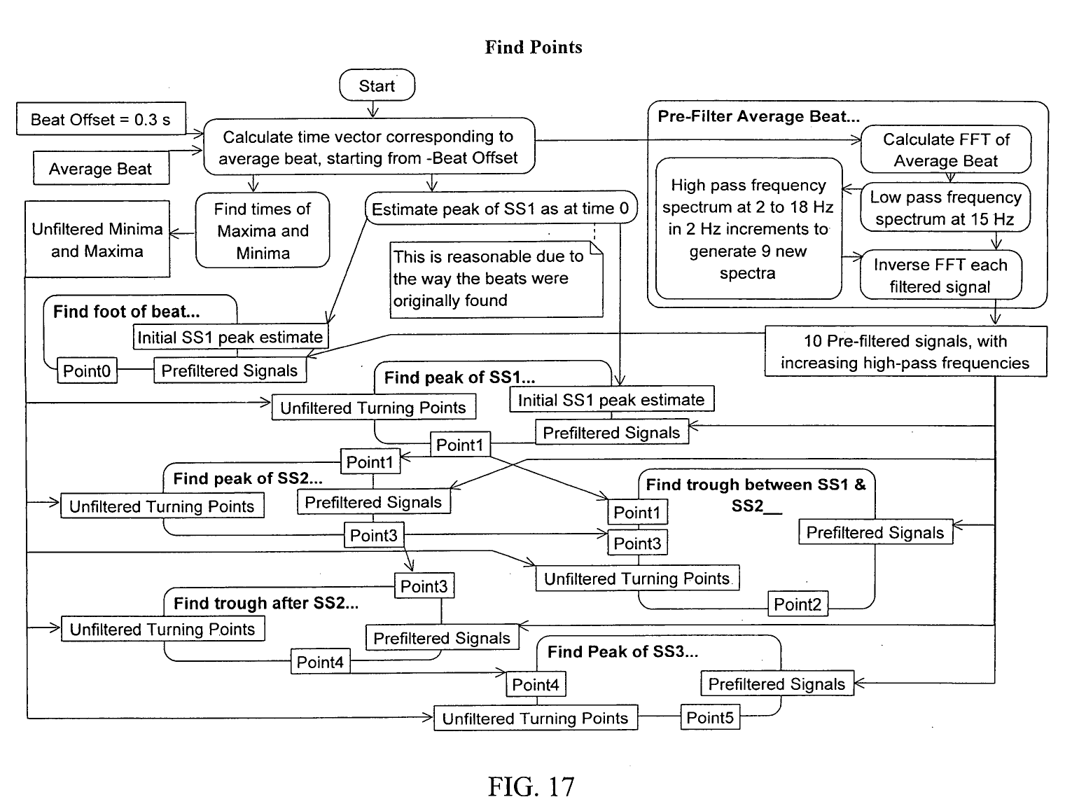

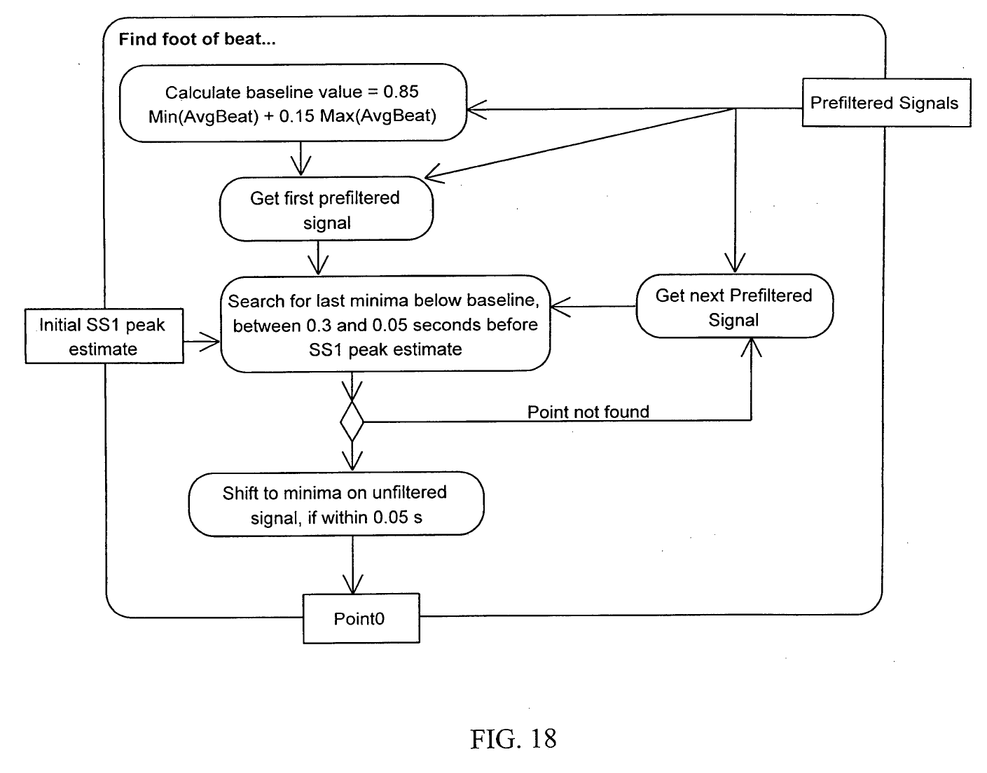

FIG. 17 is a flow chart showing the algorithm for finding feature points on the averaged pulse waveform (i.e., points a, b, c, d and e on the waveform shown in FIG. 3). Details of various portions of this algorithm are illustrated in FIGS. 18-23, respectively.

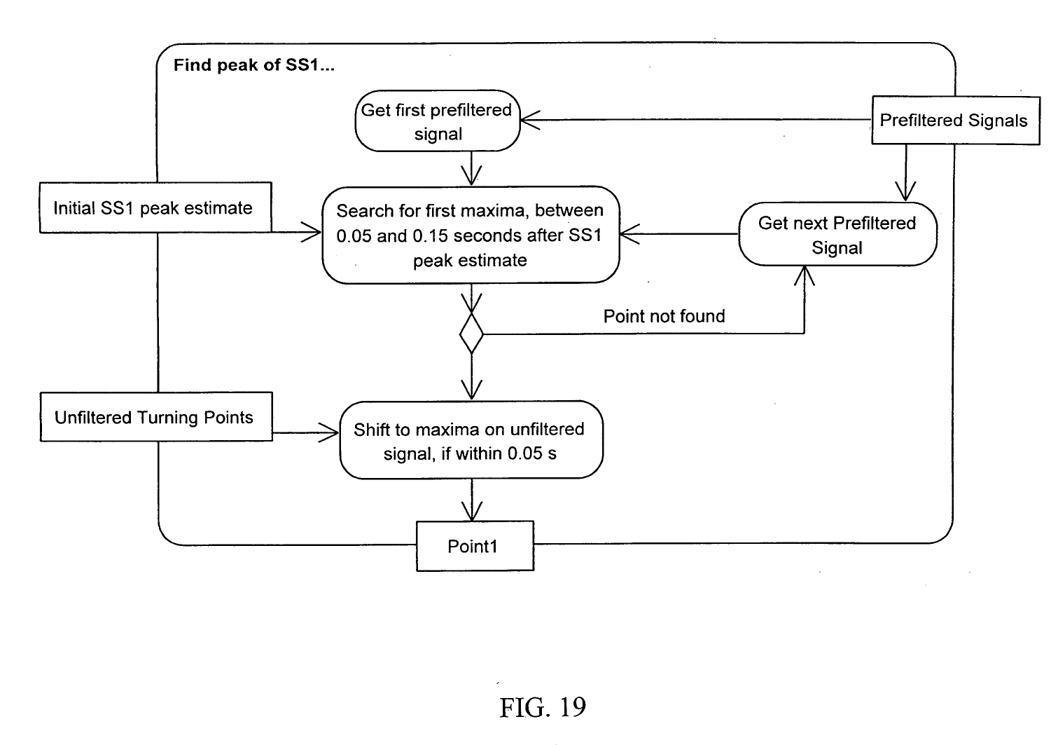

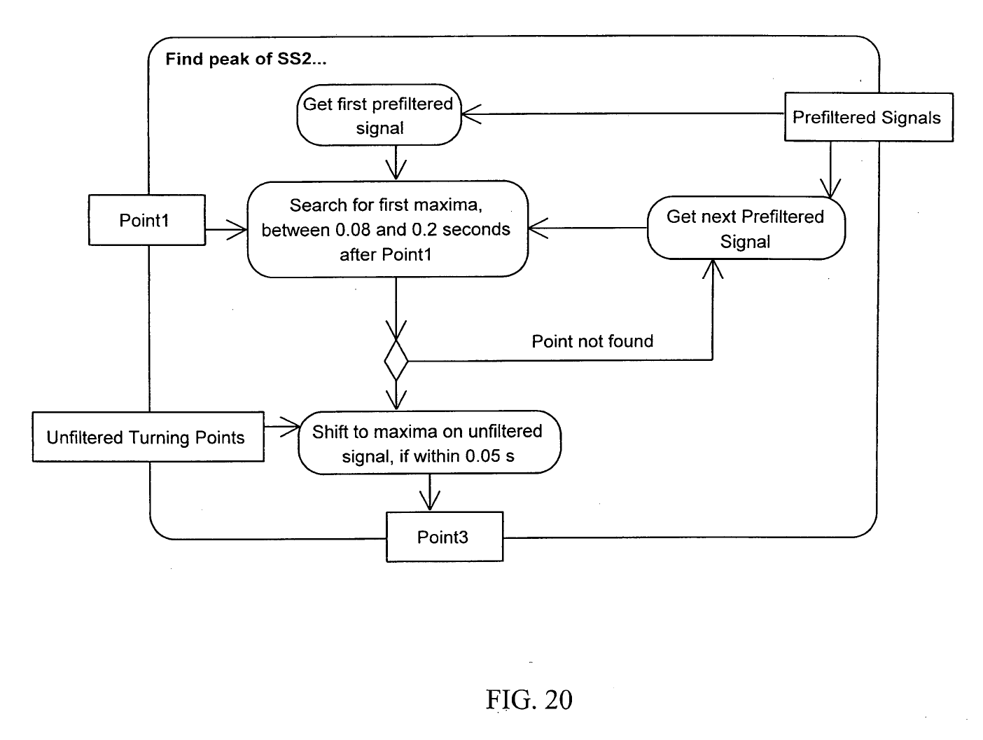

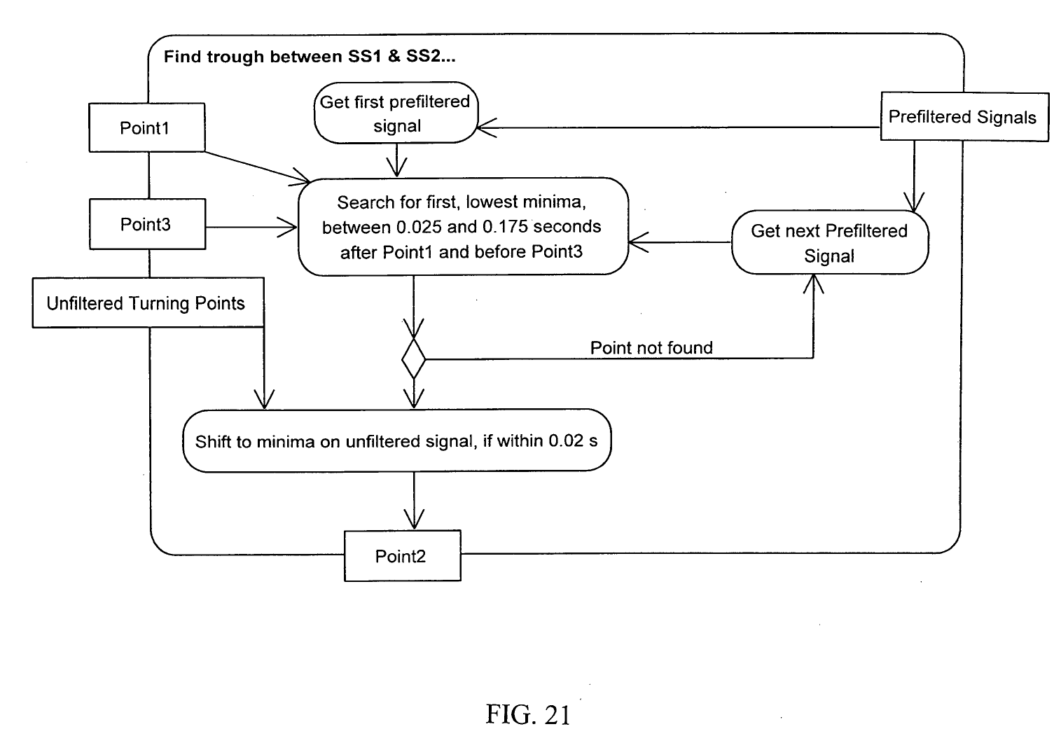

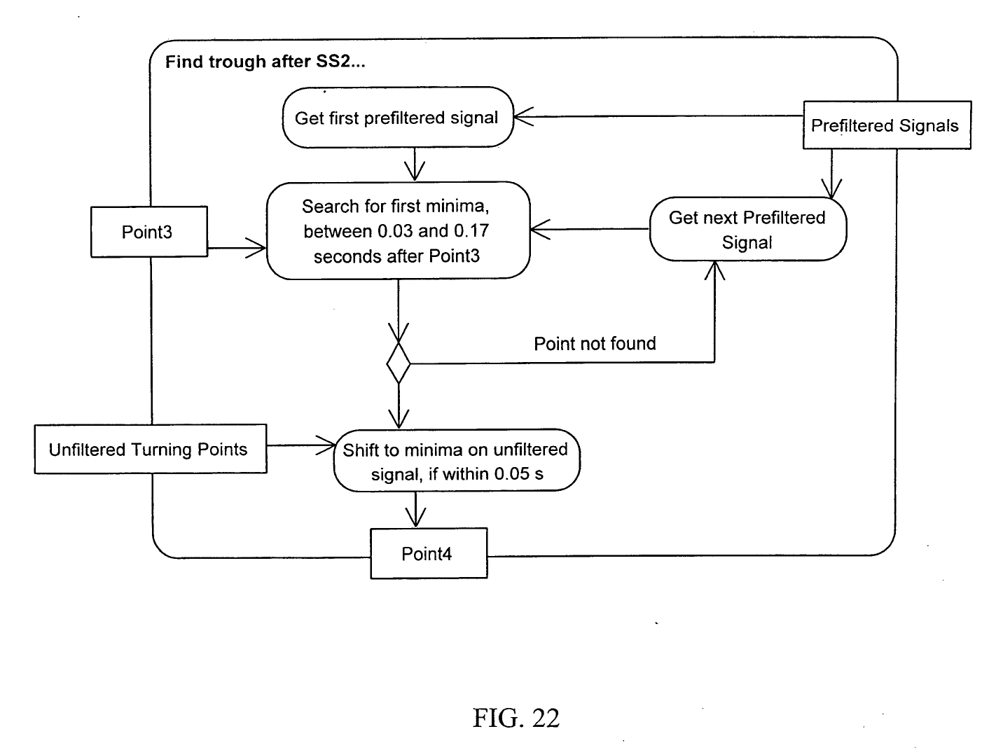

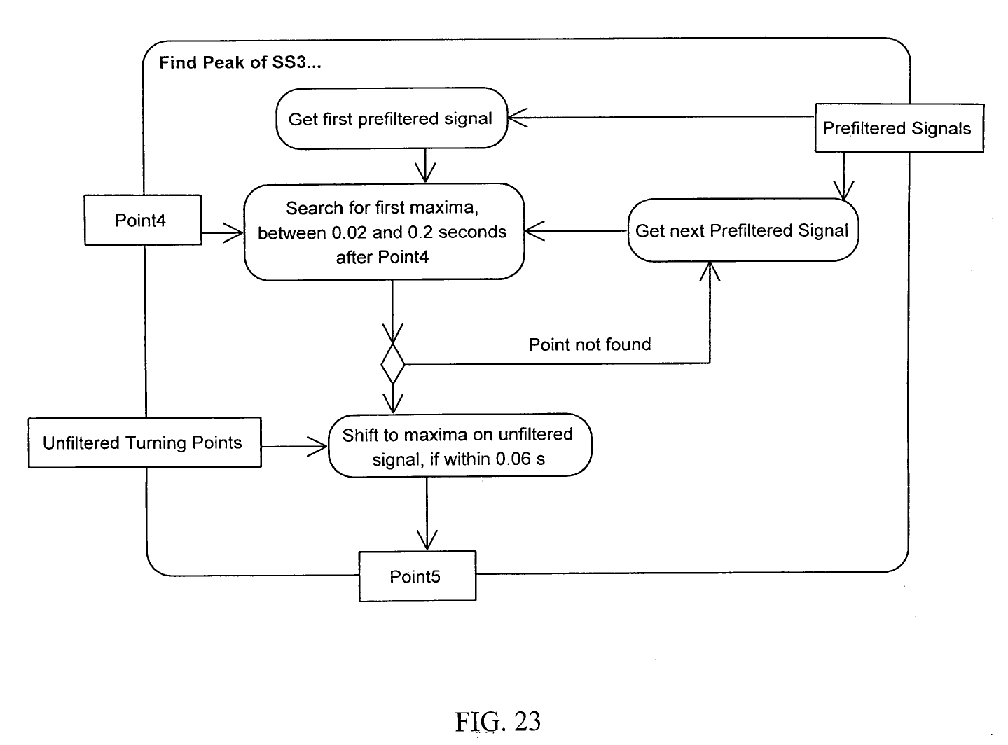

Feature points on the “average beat” are found as follows:
1. A set of pre-filtered signals is generated from the average beat, each signal being band-passed with progressively higher lower corner frequency (FIG. 17). Increasing the lower corner frequency gradually reveals inflection points that may be hidden in the original signal. The method has advantages over using first order or higher derivatives to find inflection points in that:
   It reveals the more significant inflection points first, meaning that less significant inflection points (e.g. caused by noise on the signal) are not presented as candidates for feature points that must then be screened out; and
   It better preserves the location in time of the inflection points. The system has been found to pick points more “naturally” (i.e. closer to what a human can do) than derivative-based methods.
2. An estimate of the location of the SS1 peak is made, based on the location of the zero crossings (FIG. 17).
3. The leading foot of the beat (Point0) is found, being the last significant minimum before the SS1 peak estimate (FIG. 18).
4. The peak of SS1 (Point1) is found, being a significant maximum after Point0 (FIG. 19).
5. The peak of SS2 (Point3) is found, being a significant maximum after Point1 (FIG. 20).
6. The trough between SS1 and SS2 (Point2) is found, being a significant minimum some time after Point1 and before Point3 (FIG. 21).
7. The trough after SS2 (Point4) is found, being a significant minimum some time after Point3 (FIG. 22).
8. The peak of SS3 (Point5) is found, being a significant minimum some time after Point4 (FIG. 23).

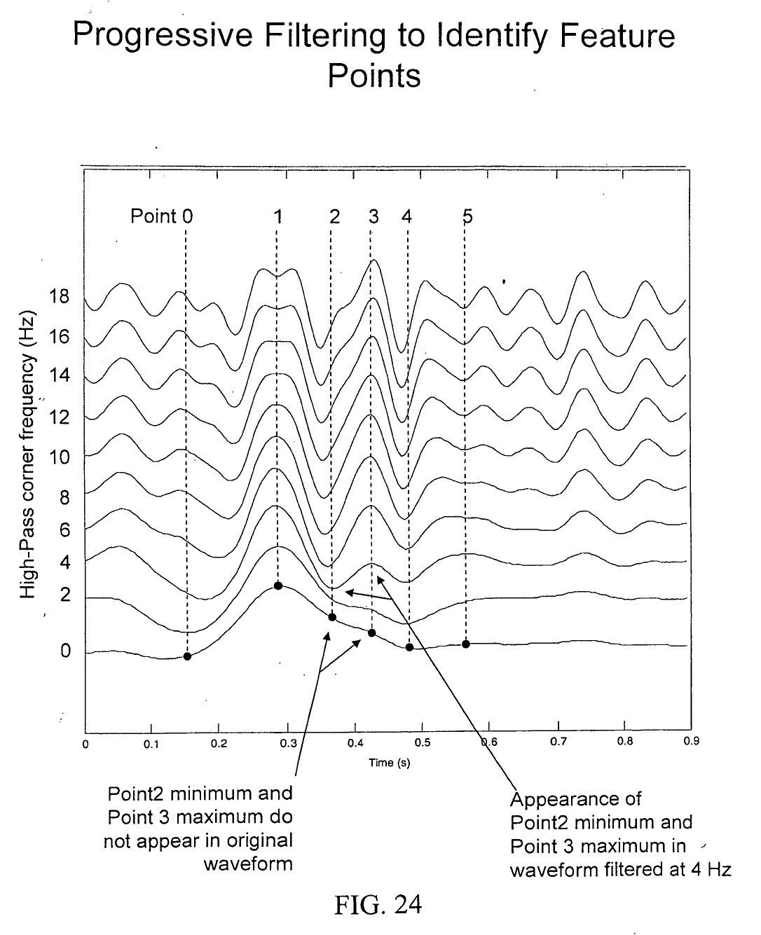

FIG. 24 is a time diagram illustrating how this process of identifying feature points is carried out by filtering at ten different, extremely low corner frequencies (0, 2, 4, 6, 8, 10, 12, 14, 16 and 18 Herz).

There has thus been shown and described a novel method and apparatus for determining cardiac medical parameters from supra-systolic signals obtained from a blood pressure cuff which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

What is claimed is:

1. A method for determining a cardiovascular status of a mammal having a cardiovascular system that includes a brachial artery, said method comprising the steps of:
(a) inflating a blood pressure cuff on the brachial artery to a supra-systolic pressure;
(b) sensing a sequence of pressure pulse waveforms associated with the brachial artery that result from a plurality of cardiac ejection cycles;
(c) averaging the waveforms to produce an average representative waveform having an initial, incident wave portion and a reflected wave portion;
(d) analyzing the representative waveform to determine a value of at least one cardiac medical parameter selected from the group consisting of the reflection wave ratio (RWR), the reflected wave transit time (RWTT), the maximum amplitude of the initial pressure wave (PS1), the maximum rise (slope) of the initial pressure wave (dp/dt), and the systolic ejection period (SEP); and
(e) displaying the value of said at least one medical parameter.

2. The method defined in claim 1, wherein the medical parameter is the reflection wave ratio.

3. The method defined in claim 1, wherein the medical parameter is the reflected wave transit time.

4. The method defined in claim 1, wherein the medical parameter is the maximum rise (slope) of the initial pressure wave.

5. The method defined in claim 1, wherein the medical parameter is the systolic ejection period.

6. The method defined in claim 1, wherein the blood pressure cuff is inflated to a pressure in the range of substantially 25 to 30 mm Hg above systole.

7. The method defined in claim 1, wherein the reflection wave ratio is proportional to the ratio of the peak amplitude of the reflected wave portion to the peak amplitude of the incident wave portion.

8. The method defined in claim 1, wherein the reflection wave transit time is the time between the commencement of the incident wave portion and the commencement of the reflected wave portion.

9. The method defined in claim 1, wherein the systolic ejection period is the time between start of the incident wave portion and the end of the reflected wave portion.

10. The method defined in claim 1, further comprising the step of applying a stress to at least a portion of the cardiovascular system of the mammal during a time period that said waveforms are sensed.

11. The method defined in claim 10, wherein the step of applying a stress includes administering a drug to the patient selected from the group consisting of a vasodilator, and a vasoconstrictor.

12. The method defined in claim 10, wherein the step of applying a stress includes administering a fluid to the patient.

13. Apparatus for determining a cardiovascular status of a mammal having a cardiovascular system that includes a brachial artery, said apparatus comprising, in combination:
(a) means for inflating a blood pressure cuff on the brachial artery to a supra-systolic pressure;
(b) means for sensing a sequence of pressure pulse waveforms associated with the brachial artery that result from a plurality of cardiac ejection cycles;
(c) means for averaging the waveforms to produce an average representative waveform having an initial, incident wave portion and a reflected wave portion;
(d) means for analyzing the representative waveform to determine a value of at least one cardiac medical parameter selected from the group consisting of the reflection wave ratio (RWR), the reflected wave transit time (RWTT), the maximum amplitude of the initial pressure wave (PS1), the maximum rise (slope) of the initial pressure wave (dp/dt), and the systolic ejection period (SEP); and
(e) means for displaying the value of said at least one medical parameter.

14. The apparatus defined in claim 13, wherein the medical parameter is the reflection wave ratio.

15. The apparatus defined in claim 13, wherein the medical parameter is the reflected wave transit time.

16. The apparatus defined in claim 13, wherein the medical parameter is the maximum rise (slope) of the initial pressure wave.

17. The apparatus defined in claim 13, wherein the medical parameter is the systolic ejection period.

18. The apparatus defined in claim 13, wherein the means for inflating inflates the cuff to a pressure in the range of substantially 25 to 30 mm Hg above systole.

19. The apparatus defined in claim 13, wherein the reflection wave ratio is determined as proportional to the ratio of the peak amplitude of the reflected wave portion to the peak amplitude of the incident wave portion.

20. The apparatus defined in claim 13, wherein the reflection wave transit time is determined as the time between the commencement of the incident wave portion and the commencement of the reflected wave portion.

21. The apparatus defined in claim 13, wherein the systolic ejection period is determined as the time between start of the incident wave portion and the end of the reflected wave portion.

22. A method of analyzing arterial blood pressure waveforms comprising the steps of:
(a) averaging the waveforms to produced an average representative waveform having an initial wave portion (SS1) and a reflected wave portion (SS2);
(b) analyzing the representative waveform to determine a time of occurrence of an initial trough, a first peak, and a second peak;
(c) determining a height of the first peak and the second peak with respect to the initial trough.

23. The method of claim 22, further comprising the steps of determining a time of occurrence of a second trough and determining a time difference between the first trough and the second trough.

24. The method of claim 22, further comprising the steps of determining a time of occurrence of a third trough and determining a time difference between the first trough and the third trough.

25. The method of claim 22, further comprising the step of determining a time of occurrence of a second trough and a third trough.

26. The method defined in claim 22, wherein step (b) comprises the steps of:
(1) pre-filtering the representative waveform to produce a plurality of waveforms, each with a different bandpass frequency; and
(2) finding troughs and peaks in said plurality of waveforms; and
(3) selecting optimum times of occurrence of the initial trough, the first peak and the second peak from among said troughs and peaks of said plurality of waveforms.
